(19) United States
(12) Patent Application Publication
(10) Pub. No.: US 2004/0176316 A1
(43) Pub. Date: Sep. 9, 2004

(54) Compounds for the normalization of the sleep/wake cycle

(76) Inventors: Perry F. Renshaw, Bedford, MA (US); Scott Lukas, Boxboro, MA (US)

Correspondence Address: CLARK & ELBING LLP, 101 FEDERAL STREET, BOSTON, MA, 02110, US

(21) Appl. No.: 10/740,075

(22) Filed: Dec. 17, 2003

Related U.S. Application Data
(60) Provisional application No. 60/435,457, filed on Dec. 20, 2002.

(51) Int. Cl. A61K 31/7072; A61K 31/7076; A61K 31/14

(52) U.S. Cl. 514/45; 514/50; 514/642

(57) ABSTRACT

The invention provides methods for normalizing the sleep/wake cycle of a mammal by administering a therapeutically-effective amount of a cytosine-containing or cytidine-containing compound, uridine-containing compound, creatine-containing compound, adenosine-containing, or adenosine-elevating compound. As such the methods described herein may increase wakefulness, reduce tiredness or fatigue during the day, and improve sleep quality. The methods of the invention may also be used in the treatment of sleep disorders, such as insomnia, sleep apnea, periodic limb movements, restless leg syndrome, narcolepsy, and problem sleepiness, or for increasing cognitive function in sleep deprived individuals. Citicoline is an exemplary compound for use in the methods described herein.

### CROSS-REFERENCE TO RELATED APPLICATIONS

This application claims benefit of priority from U.S. Provisional Application No. 60/435,457, filed Dec. 20, 2002, hereby incorporated by reference.

### BACKGROUND OF THE INVENTION

This invention relates to methods for the normalization of the sleep/wake cycle and for treatment of sleep disorders.

Sleep disorders, such as sleep apnea, insomnia, narcolepsy, restless leg syndrome, periodic limb movements, and problem sleepiness, affect numerous people of all age groups. In addition, certain compounds when used, or abused, may interrupt healthy sleeping patterns. Such compounds include stimulants, e.g., caffeine and cocaine, and depressants, e.g., alcohol. Individuals suffering from sleep disorders may experience problems concentrating or staying awake, which may interfere with work and social activities and limit the ability of the sufferer to operate motor vehicles or other machinery. Lack of adequate sleep may also weaken the immune system or alter other normal bodily functions, which may in turn lead to other conditions or illnesses.

Therefore, it would be beneficial to provide pharmacotherapies suitable for administration to all populations, including the elderly and children, for the normalization of the sleep/wake cycle and the treatment of sleep disorders.

### SUMMARY OF THE INVENTION

In general, the invention features a method of normalizing the sleep/wake cycle of a mammal by administering a therapeutically-effective amount of a cytidine-containing, cytosine-containing, creatine-containing, uridine-containing, adenosine-containing, or adenosine-elevating compound to the mammal. The methods may be used, for example, to reduce fatigue or tiredness, to increase wakefulness during the day, or to improve the sleep quality of mammals.

In a related aspect, the invention features a method of treating a sleep disorder by administering a therapeutically-effective amount of a cytidine-containing, cytosine-containing, creatine-containing, uridine-containing, adenosine-containing, or adenosine-elevating compound to a mammal. Exemplary sleep disorders include insomnia, constructive or obstructive sleep apnea, restless leg syndrome, periodic limb movements, problem sleepiness, or narcolepsy. The mammal suffering from a sleep disorder may also be suffering from a substance abuse disorder, e.g., alcohol, caffeine, or cocaine dependence or usage.

The invention further features a method of increasing cognitive function in a mammal suffering from sleep deprivation by administering a therapeutically-effective amount of a cytidine-containing, cytosine-containing, creatine-containing, uridine-containing, adenosine-containing, or adenosine-elevating compound to the mammal.

Any of the cytidine-containing, cytosine-containing, creatine-containing, uridine-containing, adenosine-containing, or adenosine-elevating compounds of the invention may be administered separately or in combination with other substances. In preferred embodiments, the cytidine-containing compound is cytidine, CDP, or CDP-choline; the cytidine-containing compound includes choline; and the mammal is a human child, adolescent, adult, or older adult. In other preferred embodiments, the CDP-choline is administered orally, and the administration is chronic, e.g., treatment occurring over a period of greater than 1, 2, 3, 4, 5, 6, 7, 14, 21, 30, 60, 90, or 180 days or even over a period of greater than one year.

In other preferred embodiments, a brain phospholipid (e.g., lecithin) or a brain phospholipid precursor (e.g., a fatty acid or a lipid), is also administered to the mammal. In other preferred embodiments, an antidepressant is also administered to the mammal.

By “sleep disorder” is meant a disorder that affects the quality, duration, or timing of a sleep pattern.

By “sleep/wake cycle” is meant the cycle of the periods in which a subject is asleep and the periods in which a subject is awake. A normal sleep/wake cycle involves sleeping at night and being awake during the day, although other sleep/wake cycles are possible, e.g., sleeping during the day and working at night.

By “sleep deprivation” is meant a lack of a normal amount of sleep. For example, adult humans sleep on average about eight hours a night, and an adult human that receives fewer than eight hours of sleep in a night is thus on average sleep deprived.

By “sleep quality” is meant a measure of the actual rest obtained from sleep, as opposed to the length of time that a mammal is asleep.

By “abuse” is meant excessive use of a substance, particularly one that may modify body functions.

By “dependence” or “dependency” is meant any form of behavior that indicates an altered or reduced ability to make decisions resulting, at least in part, from the use of a substance. Representative forms of dependency behavior may take the form of antisocial, or inappropriate behavior and include those behaviors directed at the desire, planning, acquiring, and use of a substance. This term also includes the psychic craving for a substance that may or may not be accompanied by a physiological dependency, as well as a state in which there is a compulsion to use a substance, either continuously or periodically, in order to experience its psychic effects or to avoid the discomfort of its absence. Forms of dependency include habituation, that is, an emotional or psychological dependence on a substance to obtain relief from tension and emotional discomfort; tolerance, that is, the progressive need for increasing doses to achieve and sustain a desired effect; addiction, that is, physical or physiological dependence which is beyond voluntary control; and use of a substance to prevent withdrawal symptoms. Dependency may be influenced by a number of factors, including physical characteristics of the user (e.g., genetic predisposition, age, gender, or weight), personality, or socioeconomic class.

By “treating” is meant the medical management of a patient with the intent that a cure, amelioration, stabilization, or prevention of a disease, pathological condition, or disorder will result. This term includes active treatment, that is, treatment directed specifically toward improvement of a disease, pathological condition, or disorder, and also includes causal treatment, that is, treatment directed toward removal of the cause of the disease, pathological condition, or disorder. In addition, this term includes palliative treatment, that is, treatment designed for the relief of symptoms rather than the curing of the disease, pathological condition, or disorder; preventive treatment, that is, treatment directed to prevention of the disease, pathological condition, or disorder; and supportive treatment, that is, treatment employed to supplement another specific therapy directed toward the improvement of the disease, pathological condition, or disorder. The term “treating” also includes symptomatic treatment, that is, treatment directed toward constitutional symptoms of the disease, pathological condition, or disorder.

By “therapeutically-effective amount” is meant an amount of a cytidine-containing, cytosine-containing compound, a uridine-containing compound, a creatine-containing compound, an adenosine-containing compound, and an adenosine-elevating compound sufficient to produce a healing, curative, prophylactic, stabilizing, or ameliorative effect in a mammal suffering from a sleep disorder, an abnormal sleep/wake cycle, or sleep deprivation.

By “cytidine-containing compound” is meant any compound that includes, as a component, cytidine, CMP, CDP, CTP, dCMP, dCDP, or dCTP. Cytidine-containing compounds can include analogs of cytidine. Preferred cytidine-containing compounds include, without limitation, CDP-choline and cytidine 5′-diphosphocholine, frequently prepared as cytidine 5′-diphosphocholine [sodium salt] and also known as citicoline.

By “cytosine-containing compound” is meant any compound that includes, as a component, cytosine. Cytosine-containing compounds can include analogs of cytosine.

By “adenosine-containing compound” is meant any compound that includes, as a component, adenosine. Adenosine-containing compounds can include analogs of adenosine.

By “adenosine-elevating compound” is meant any compound that elevates brain adenosine levels, for example, compounds which inhibit or alter adenosine transport or metabolism (e.g., dipyridamole or S-adenosylmethionine).

By “uridine-containing compound” is meant any compound that includes as a component, uridine or UTP. Uridine-containing compounds can include analogs of uridine, for example, triacetyl uridine.

By “creatine-containing compound” is meant any compound that includes as a component, creatine. Creatine-containing compounds can include analogs of creatine.

By “phospholipid” is meant a lipid containing phosphorus, e.g., phosphatidic acids (e.g., lecithin), phosphoglycerides, sphingomyelin, and plasmalogens. By “phospholipid precursor” is meant a substance that is built into a phospholipid during synthesis of the phospholipid, e.g., fatty acids, glycerol, or sphingosine.

By “child or adolescent” is meant an individual who has not attained complete growth and maturity. Generally, a child or adolescent is under twenty-one years of age.

By “older adult” is meant an individual who is in the later stage of life. Generally, an older adult is over sixty years of age.

The compounds utilized herein are relatively non-toxic, and CDP-choline, uridine, and triacetyl uridine, in particular, are pharmacokinetically understood and known to be well tolerated by mammals. The present invention, therefore, provides treatments that are likely to have few adverse effects and may be administered to children and adolescents, as well as the elderly, or those whose health is compromised because of existing physical conditions.

Other features and advantages will be apparent from the following description and the claims.

### BRIEF DESCRIPTION OF THE DRAWINGS

FIG. 1 is a graph of the effects of citicoline on sleep quality and mood.

FIGS. 2A and 2B are graphs of level of activity as a function of time of day without (A) and with (B) citicoline treatment. A, B, and C refer to use of alcohol, caffeine, and cocaine, respectively. The numbers indicate a craving for cocaine, based on a 10 point scale. Gray dots indicate ingestion of citicoline.

FIG. 3 is a schematic illustration of the molecular structure of CDP-choline.

### DETAILED DESCRIPTION OF THE INVENTION

The invention described herein features methods for the normalization of the sleep/wake cycle, for treatment of sleep disorders, and for increasing cognitive functioning in sleep deprived mammals. The impact of such normalization may lead to an improvement in the “sleep quality” that is perceived by the individual. To this end, the invention features the use of cytidine-containing, cytosine-containing, uridine-containing, creatine-containing, adenosine-containing, and adenosine-elevating compounds to effect a desired outcome. A preferred cytidine-containing compound is CDP-choline (also referred to as citicoline or CDP choline [sodium salt]), a preferred adenosine-containing compound is S-adenosylmethionine (SAMe), and a preferred uridine-containing compound is triacetyl uridine.

The cytidine-containing, cytosine-containing, uridine-containing, creatine-containing, adenosine-containing, or adenosine-elevating compounds may be co-administered with other compounds, such as precursors for the synthesis of brain phospholipids, e.g., fatty acids, lipids, or lecithin.

Sleep/Wake Cycle

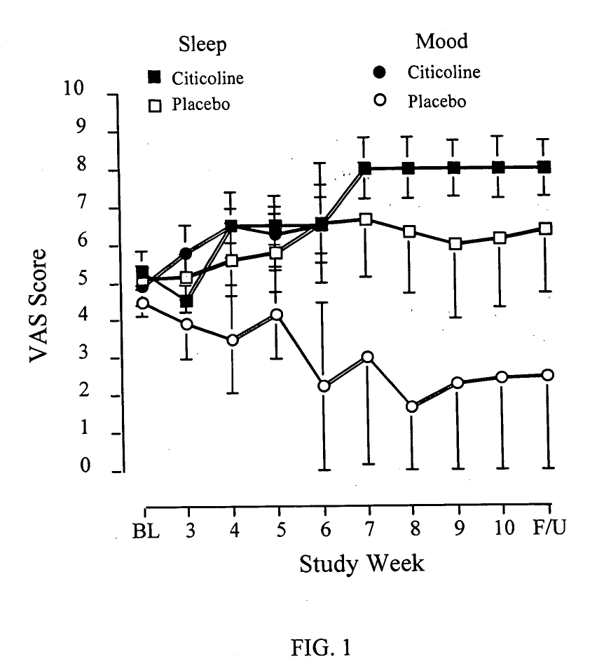

Surprisingly, we have discovered that citicoline (CDP-choline) is useful for the normalization of the sleep/wake cycle. The quality of sleep is improved, and the sleep/wake cycle is normalized after 2-4 weeks of citicoline treatment. This normalization of the sleep/wake cycle may further promote increased wakefulness or reduce fatigue or tiredness during the day. The administration of citicoline will also likely stabilize the homeostatic processes involved in sleep disorders such as insomnia, sleep apneas (central or obstructive), problem sleepiness, restless leg syndrome, periodic limb movements, and narcolepsy. In addition, citicoline may increase cognitive functioning (Alvarez et al. Methods Find Exp Clin Pharmacol 21:633-44, 1999; Fioravanti et al. Cochrane Database Syst. Rev. 4: CD000269, 2000) and may be used to increase cognitive performance in individuals in a sleep-deprived state, e.g., pilots, physicians, students, or others who may experience long periods without sleep. Data in FIG. 1 show that the administration of citicoline increases the quality of sleep and mood of human subjects, as measured by the subjects on a 10 point scale, compared to subjects receiving a placebo. Since CDP-choline is rapidly metabolized to cytidine and choline after administration, and cytidine is converted to uridine, the administration of any of these compounds may have a beneficial effect.

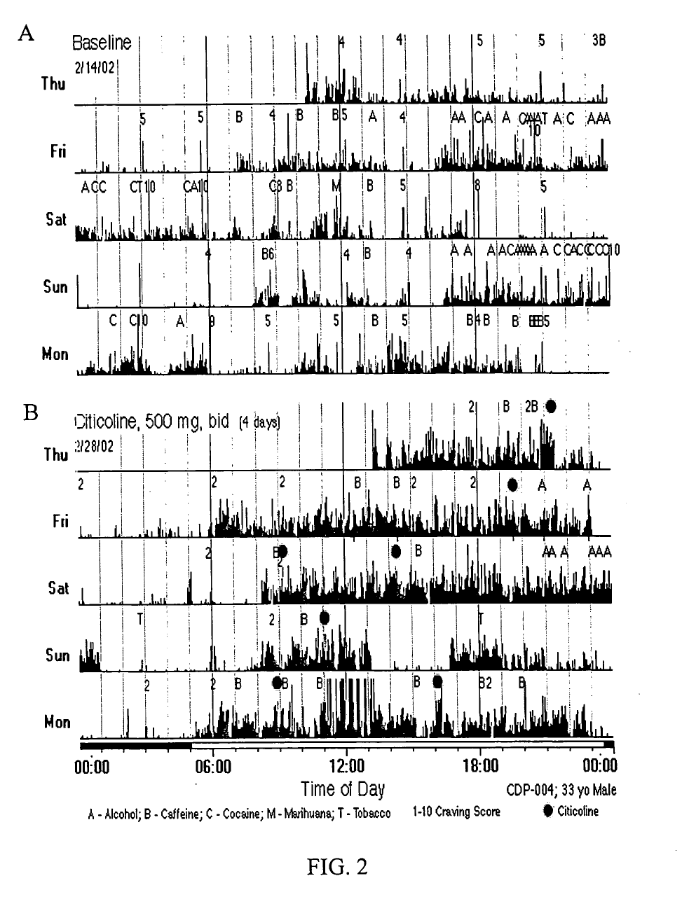

CDP-choline and related compounds are also useful in the treatment of substance abuse disorders, such as alcohol, cocaine, opiate, opioid, nicotine, or tobacco usage or dependence (U.S. Pat. Nos. 5,958,896 and 6,103,703 and U.S. Provisional Application No. 60/424,972, filed Nov. 8, 2002). Since substance abuse disorders may cause a disruption in the quality of sleep or the sleep/wake cycle, the methods of the invention may be used to normalize the sleep/wake cycle or treat sleep disorders in patients with a substance abuse disorder. In addition, a substance abuse disorder and an abnormal sleep/wake cycle or sleep disorder may be treated simultaneously with the methods described herein. FIGS. 2A and 2B show data on the level of activity of a cocaine user for five days without treatment (FIG. 2A) and for 5 days after treatment with CDP-choline (FIG. 2B, monitoring began 4 days after treatment). The sleep/wake cycle of the subject was normalized to a diurnal pattern after treatment, and the subject was more active during the day. In addition, the subject's use of cocaine (denoted by C) was eliminated with treatment, and the use of alcohol (denoted by A) was reduced. Cravings for cocaine were also reduced in intensity (denoted by numbers) after treatment.

Cytidine-Containing and Cytosine-Containing Compounds

Useful cytidine-containing or cytosine-containing compounds may include any compound comprising one of the following: cytosine, cytidine, CMP, CDP, CTP, dCMP, dCDP, and dCTP. Preferred cytidine-containing compounds include CDP-choline and cytidine 5′-diphosphocholine [sodium salt]. This list of cytidine-containing and cytosine-containing compounds is provided to illustrate, rather than to limit the invention, and the compounds described above are commercially available, for example, from Sigma Chemical Company (St. Louis, Mo.).

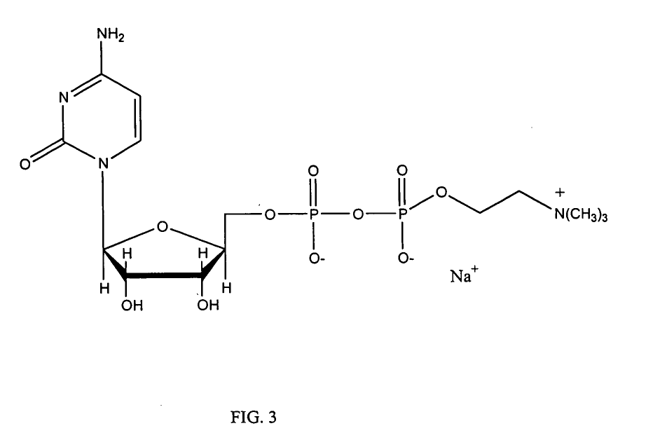

CDP-choline is a naturally occurring compound that is hydrolyzed into its components of cytidine and choline in vivo. CDP-choline is synthesized from cytidine-5′-triphosphate and phosphocholine with accompanying production of inorganic pyrophosphate in a reversible reaction catalyzed by the enzyme CTP:phosphocholine cytidylyltransferase (Weiss, Life Sciences 56:637-660, 1995). CDP-choline is available for oral administration in a 500 mg oblong tablet. Each tablet contains 522.5 mg CDP-choline sodium, equivalent to 500 mg of CDP-choline. Matching placebo tablets are also available. The excipients contained in both active and placebo tablets are talc, magnesium stearate, colloidal silicon dioxide, hydrogenated castor oil, sodium carboxymethylcellulose, and microcrystalline cellulose. The molecular structure of CDP-choline [sodium salt] is provided in FIG. 3.

Other formulations for treatment or of sleep disorders may take the form of a cytosine-containing or cytidine-containing compound combined with a pharmaceutically-acceptable diluent, carrier, stabilizer, or excipient.

### Adenosine-Containing and Adenosine-Elevating Compounds

Adenosine-containing or adenosine-elevating compounds also provide useful therapies. Data from animal tests show that administration of adenosine analogs increases the amount of slow wave sleep (Radulovacki M et al. J Pharmacol Exp Ther 228:268-74, 1984; Satoh S et al. Eur J Pharmacol 351:155-62, 1998; Scammell T E et al. Neuroscience 107:653-63, 2001). In addition, magnetic resonance data indicate that sleep deprivation leads to a build up of adenosine. This build up may be the neurobiological basis of “sleep pressure,” and this build up of adenosine may then allow for recovery sleep. Thus, these compounds may play an integral role in the maintenance of sleep homeostasis.

Useful adenosine-containing or adenosine-elevating compounds include, without limitation, any compound comprising one of the following adenosine, ATP, ADP, or AMP. One preferred adenosine-containing compound is S-adenosylmethionine (SAMe).

In addition, compounds are known that are capable of increasing adenosine levels by other mechanisms. For example, adenosine uptake can be inhibited by a number of known compounds, including propentofylline (described in U.S. Pat. No. 5,919,789). Another known compound that inhibits adenosine uptake is EHNA.

Other useful compounds that can be used to increase brain adenosine levels are those that inhibit enzymes that break down adenosine, (e.g., adenosine deaminase and adenosine kinase). Finally, administering compounds that contain adenosine or precursors of adenosine, which are released as adenosine in vivo, can also be used.

Uridine-Containing Compounds

Uridine and uridine-containing compounds provide useful therapies because these compounds can be converted to CTP, a rate-limiting factor in PC biosynthesis (Wurtman et al., Biochemical Pharmacology 60:989-992, 2000). Useful uridine-containing compounds include, without limitation, any compound comprising uridine, UTP, UDP, or UMP. A preferred uridine-containing compound is triacetyl uridine. Uridine and uridine-containing compounds and analogs are well tolerated in humans.

Creatine-Containing Compounds

Creatine and creatine-containing compounds provide useful therapies because these compounds, by virtue of increasing brain phospholipid levels, can raise the levels of ATP. Creatine and creatine-containing compounds are known to be well tolerated at relatively high doses in humans.

Administration

Conventional pharmaceutical practice is employed to provide suitable formulations or compositions for administration to patients. Oral administration is preferred, but any other appropriate route of administration may be employed, for example, parenteral, intravenous, subcutaneous, intramuscular, intracranial, intraorbital, ophthalmic, intraventricular, intracapsular, intraspinal, intracisternal, intraperitoneal, intranasal, or aerosol administration. Therapeutic formulations may be in the form of liquid solutions or suspensions (as, for example, for intravenous administration); for oral administration, formulations may be in the form of liquids, tablets, or capsules; and for intranasal formulations, in the form of powders, nasal drops, or aerosols.

Methods well known in the art for making formulations are described, for example, in Remington: The Science and Practice of Pharmacy (20th ed.) ed. A. R. Gennaro, 2000, Lippincott, Philadelphia, Pa. Formulations for parenteral administration may, for example, contain excipients, sterile water, saline, polyalkylene glycols such as polyethylene glycol, oils of vegetable origin, or hydrogenated napthalenes.

If desired, slow release or extended release delivery systems may be utilized. Biocompatible, biodegradable lactide polymer, lactide/glycolide copolymer, or polyoxyethylene-polyoxypropylene copolymers may be used to control the release of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Preferably, the compounds of the invention, such as CDP-choline, are administered at a dosage of at least 500 mg twice daily by oral administration. Orally administered CDP-choline is bioavailable, with more than 99% of CDP-choline and/or its metabolites absorbed and less than 1% excreted in feces. CDP-choline, administered either orally or intravenously, is rapidly converted into the two major circulating metabolites, choline and cytidine. Major excretion routes are lung (12.9%) and urine (2.4%); the rest of the dose (83.9%) is apparently metabolized and retained in tissues.

In general, the compounds of the invention, such as CDP-choline, uridine, UTP, creatine, or SAMe, are administered at a dosage appropriate to the effect to be achieved and are typically administered in unit dosage form. The dosage preferably ranges from 50 mg per day to 2000 mg per day. The exact dosage of the compound may be dependent, for example, upon the age and weight of the recipient, the route of administration, and the severity and nature of the symptoms to be treated. In general, the dosage selected should be sufficient to treat the sleep disorder, or one or more symptoms thereof, without producing significant toxic or undesirable side effects. As noted above, the preferred route of administration for most indications is oral.

In the case of CDP-choline, there have been no reported cases of overdoses. CDP-choline toxicity is largely self-limiting, ingestion of large amounts in preclinical studies shows common cholinergic symptoms (salivation, lacrimation, urination, defecation, and vomiting).

Combination With Other Therapeutics

The cytidine-containing, cytosine-containing, uridine-containing, creatine-containing, adenosine-containing, and adenosine-elevating compounds of the invention may be administered as a monotherapy, in combination with each other, or in combination with other compounds for the treatment of abnormal sleep/wake cycles or sleep disorders or other associated physiological or psychological conditions.

The compounds of the invention, may be administered in conjunction with lower doses of current treatments for these disorders, including antidepressants. For example, the compounds of the invention may be administered with phospholipids, e.g., lecithin, or with brain phospholipid precursors, e.g., fatty acids or lipids, or may be administered as an adjunct to standard therapy.

In one particular example, the compound of the invention may be administered in combination with an antidepressant, anticonvulsant, antianxiety, antimanic, antipyschotic, antiobsessional, sedative-hypnotic, or anti-hypertensive medication. Examples of these medications include, but are not limited to, the antianxiety medications, alprazolam, buspirone hydrochloride, chlordiazepoxide, chlordiazepoxide hydrochloride, clorazepate dipotassium, desipramine hydrochloride, diazepam, halazepam, hydroxyzine hydrochloride, hydroxyzine pamoate, lorazepam, meprobamate, oxazepam, prazepam, prochlorperazine maleate, prochlorperazine, prochlorperazine edisylate, and trimipramine maleate; the anticonvulsants, amobarbital, amobarbital sodium, carbamazepine, chlordiazepoxide, chlordiazepoxide hydrochloride, clorazepate dipotassium, diazepam, divalproex sodium, ethosuximide, ethotoin, gabapentin, lamotrigine, magnesium sulfate, mephenytoin, mephobarbital, methsuximide, paramethadione, pentobarbital sodium, phenacemide, phenobarbital, phenobarbital sodium, phensuximide, phenytoin, phenytoin sodium, primidone, secobarbital sodium, trimethadione, valproic acid, and clonazepam; the antidepressants, amitriptyline hydrochloride, amoxapine, bupropion hydrochloride, clomipramine hydrochloride, desipramine hydrochloride, doxepin hydrochloride, fluoxetine, fluvoxamine, imipramine hydrochloride, imipramine pamoate, isocarboxazid, lamotrigine, maprotoline hydrochloride, nortriptyline hydrochloride, paroxetine hydrochloride, phenelzine sulfate, protriptyline hydrochloride, sertraline hydrochloride, tranylcypromine sulfate, trazodone hydrochloride, trimipramine maleate, and venlafaxine hydrochloride; the antimanic medications, lithium carbonate and lithium citrate; the antiobsessional medications, fluvoxamine, and clomipramine hydrochloride; the antipsychotic medications, acetophenazine maleate, chlorpromazine hydrochloride, chlorprothixene, chlorprothixene hydrochloride, clozapine, fluphenazine decanoate, fluphenazine enathrate, fluphenazine hydrochloride, haloperidol decanoate, haloperidol, haloperidol lactate, lithium carbonate, lithium citrate, loxapine hydrochloride, loxapine succinate, mesoridazine besylate, molindone hydrochloride, perphenazine, pimozide, prochlorperazine maleate, prochlorperazine, prochlorperazine edisylate, promazine hydrochloride, risperidone, thioridazine, thioridazine hydrochloride, thiothixene, thiothixene hydrochloride, and trifluoperzine hydrochloride; the sedative-hypnotic medications, amobarbital, amobarbital sodium, aprobarbital, butabarbital, chloral hydrate, chlordiazepoxide, chlordiazepoxide hydrochloride, clorazepate dipotassium, diazepam, diphenhydramine, estazolam, ethchlorvynol, flurazepam hydrochloride, glutethimide, hydroxyzine hydrochloride, hydroxyzine pamoate, lorazepam, methotrimeprazine hydrochloride, midazolam hydrochloride, non prescription, oxazepam, pentobarbital sodium, phenobarbital, phenobarbital sodium, quazepam, secobarbital sodium, temazepam, triazolam, and zolpidem tartrate; and the anti-hypertensive, clonidine.

### OTHER EMBODIMENTS

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the appended claims.

Other embodiments are within the claims.

What is claimed is:

1. A method of normalizing the sleep/wake cycle of a mammal, said method comprising administering a therapeutically-effective amount of a compound selected from the group consisting of a cytidine-containing compound, a cytosine-containing compound, a uridine-containing compound, a creatine-containing compound, an adenosine-containing compound, and an adenosine-elevating compound to a mammal, thereby normalizing the sleep/wake cycle of said mammal.

2. The method of claim 1, wherein said administration reduces fatigue or tiredness, increases wakefulness, or improves the sleep quality of said mammal during the day.

3. The method of claim 1, wherein said cytidine-containing compound is cytidine.

4. The method of claim 1, wherein said cytidine-containing compound further comprises choline.

5. The method of claim 1, wherein said cytidine-containing compound is CDP-choline.

6. The method of claim 5, wherein said CDP-choline is administered orally.

7. The method of claim 1, wherein said cytidine-containing compound is CDP.

8. The method of claim 1, wherein said administering is chronic.

9. The method of claim 1, wherein said mammal is a human.

10. The method of claim 9, wherein said human is a child or adolescent.

11. The method of claim 9, wherein said human is an older adult.

12. A method of treating a sleep disorder, said method comprising administering to a mammal a therapeutically-effective amount of a compound selected from the group consisting of a cytidine-containing compound, a cytosine-containing compound, a uridine-containing compound, a creatine-containing compound, an adenosine-containing compound, and an adenosine-elevating compound.

13. The method of claim 12, wherein said sleep disorder is caused by a substance abuse disorder.

14. The method of claim 13, wherein said substance abuse disorder is alcohol, caffeine, or cocaine usage or dependence.

15. The method of claim 12, wherein said sleep disorder is insomnia, constructive or obstructive sleep apnea, restless leg syndrome, periodic limb movements, problem sleepiness, or narcolepsy.

16. The method of claim 12, wherein said cytidine-containing compound is CDP-choline.

17. A method of increasing cognitive function, said method comprising administering a therapeutically-effective amount of a compound selected from the group consisting of a cytidine-containing compound, a cytosine-containing compound, a uridine-containing compound, a creatine-containing compound, an adenosine-containing compound, and an adenosine-elevating compound to a mammal suffering from sleep deprivation, thereby increasing the cognitive functioning of said mammal.

18. The method of claim 17, wherein said cytidine-containing compound is CDP-choline.
